SEQUENCE LISTING (1 sequence)

SEQ ID NO: 1 - protein, 126 aa, Artificial Sequence
  Sythetic Peptide
MAMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSD

(19) United States
(12) Patent Application Publication
(10) Pub. No.: US 2016/0271218 A1
(43) Pub. Date: Sep. 22, 2016

(54) SOLUBLE CTLA-4 MOLECULES AND DERIVATIVES THEREOF FOR TREATMENT OF MINIMAL CHANGE DISEASE

(71) Applicant: Alexander BIRO, Tel Aviv (IL)

(72) Inventor: Alexander Biro, Tel Aviv (IL)

(73) Assignee: Mor Research Applications Ltd., Tel Aviv (IL)

(21) Appl. No.: 14/901,082

(22) PCT Filed: Jun. 26, 2014
(86) PCT No.: PCT/IL2014/050574

§ 371 Date: Dec. 28, 2015

Related U.S. Application Data
(60) Provisional application No. 61/839,882, filed on Jun. 27, 2013.

(51) Int. Cl. A61K 38/17; A61K 47/48; A61K 45/06; C07K 14/705

(52) U.S. Cl. CPC A61K 38/1774; C07K 14/70521; A61K 47/48561; A61K 45/06; C07K 2319/32; C07K 2319/30; C07K 2319/74

(57) ABSTRACT

This disclosure relates to compositions and methods for treatment of minimal change disease. The compositions include soluble CTLA-4 molecules, and derivatives thereof, including belatacept and belatacept-like molecules that have been conjugated to a human immunoglobulin constant domain fragment.

### CROSS REFERENCE TO RELATED APPLICATIONS

Benefit is claimed to U.S. Provisional Patent Application No. 61/839,882, filed Jun. 27, 2013; the contents of which are incorporated by reference herein in their entirely.

### FIELD

This disclosure relates to compositions and methods for treatment of minimal change disease. The compositions include soluble CTLA-4 molecules, and derivatives thereof, including belatacept and belatacept-like molecules that have been conjugated to a human immunoglobulin constant domain fragment.

### BACKGROUND

Minimal change disease (MCD) is a major cause of nephrotic syndrome in both children and adults. MCD and focal segmental glomerulosclerosis (FSGS) are podocytopathies or glomerular diseases that are defined by primary lesions of the podocyte or glomerular epithelial cells. MCD symptoms include increased protein concentration in urine, and edema.

The exact pathogenesis of MCD is unclear. The disease is considered to be a disorder of T cell function. A circulating cytokine, IL13, has been postulated to link between proteinuria and T cell dysfunction. Current treatment involves prescription of corticosteroid, optionally in combination with angiotensin-converting-enzyme (ACE) inhibitors. In spite of the current treatment for primary MCD, in about 75% of the patients the symptoms recur in what in is known as a relapse. Thus, a continuing need exists for compositions that can be used to treat MCD.

### SUMMARY

Described herein is an active agent comprising a molecule which binds CD80 and/or CD86, for use in treatment of minimal change disease, wherein said molecule comprises:(i) a soluble extracellular domain of CTLA4; (ii) a mutated soluble extracellular domain of CTLA4, wherein (a) an alanine at position 29 is substituted with an amino acid selected from the group consisting of tyrosine, leucine, tryptophan, and threonine, and (b) a leucine at position 104 is substituted with a glutamic acid; or (iii) the soluble extracellular domain of (i) or (ii), and a moiety which alters the solubility and/or affinity to CD80 and/or CD86 of the extracellular domain of CTLA4. Compositions comprising the active agent and optionally other agents for treatment of MCD are also described.

Additionally described herein are methods of treating MCD, including primary and relapse MCD in a subject by administering to a subject in need thereof a composition comprising the active agents described herein. Such treatments can be administered in particular embodiments with standard MCD treatments, including steroid treatments, either in concomitantly or sequentially; in a single composition, or in multiple compositions.

The foregoing and other objects, features, and advantages will become more apparent from the following detailed description, which proceeds with reference to the accompanying figures.

### BRIEF DESCRIPTION OF THE DESCRIBED SEQUENCES

The nucleic and/or amino acid sequences provided herewith are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file named Mor_MCD_seq.txt, created June 26, 204, about 2 KB, which is incorporated by reference herein. In the accompanying sequence listing:

SEQ ID NO: 1 is the amino acid sequence of the belatacept peptide, as described herein.

### DETAILED DESCRIPTION

### I. Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

In case of conflict, the present specification, including explanations of terms, will control. In addition, all the materials, methods, and examples are illustrative and not intended to be limiting.

II. Compositions and Methods for Treatment of MCD CD80 (also known as B7-1) is a transmembrane protein that provides a costimulatory signal for T cell activation. It works in tandem with CD86 (also known as B7-2) to prime T cells. T regulatory cells secrete soluble CTLA-4 (sCTLA-4), which binds CD80 and block the co-stimulatory activation of T cells. CD80 expression has been shown in experimental models of nephrotic syndrome for example in aminonucleoside nephrosis. CD80 expression has also been related to MCD (Reiser et al., J Am Soc Nephrol, 15: 2246-2248, 2004; and Garin et al., J Am Soc Nephrol, 20: 260-266, 2009).

Provided herein are compositions for treatment of minimal change disease (MCD) and that block the positive T cell co-stimulatory pathway comprising, as an active component, a molecule which binds, or is capable of binding, CD80 and/or CD86, wherein said molecule includes: (i) a soluble extracellular domain of CTLA4; (ii) a mutated soluble extracellular domain of CTLA4, wherein (a) an alanine at position 29 is substituted with an amino acid selected from the group consisting of tyrosine, leucine, tryptophan, and threonine, and (b) a leucine at position 104 is substituted with a glutamic acid; or (iii) the soluble extracellular domain of (i) or (ii) and a moiety which alters the solubility and/or affinity to CD80 and/or CD86 of the extracellular domain of CTLA4.

Methods of using the described composition by administering a therapeutically effect amount to a subject in need thereof are also described.

For clarity, the term “the molecule which binds, or is capable of binding, CD80 and/or CD86” as defined herein above is used herein interchangeably with the term “belatacept-like molecule”, which should be understood to include also “belatacept” itself and abatacept.

Abatacept (CTLA4-Ig), an agent approved for use in rheumatoid and polyarticular juvenile idiopathic arthritis, was developed to inhibit the B7 ligands found on antigen presenting cells (APCs), which binds to CD28, a transmembrane protein required for T-cell activation and proliferation. This agent was assessed in animal models of organ transplant. After initial failures, a more potent derivative of abatacept, belatacept, was developed.

Belatacept (also described herein as the amino acid sequence set forth as SEQ ID NO: 1) is the result of altering two amino acids in the CD80/86 binding portion of the abatacept compound (L104E and A29Y; Due to ambiguity regarding the site of cleavage of the leader sequence, the extracellular domain may start with either a Methionine or an Alanine. Unless context clearly dictates otherwise, the numbering system used herein is that wherein position 1 is Met). This slight change in chemistry resulted in a 10-fold increase in the ability to inhibit T-cell activation when compared in vitro. As described herein, the moiety which alters the solubility and/or affinity to CD80 and/or CD86 of the extracellular domain of CTLA4 can be a non-proteinaceous moiety such as polyethylene glycol (PEG), or it can be a fraction of an immunoglobulin molecule, such as the Fc, constant region, of an IgG.

In certain embodiments, the moiety comprises a human immunoglobulin constant region.

In certain embodiments, the belatacept-like molecule comprises the amino acid sequence of belatacept having the amino acid sequence of SEQ ID NO: 1.

As stated above, current treatment involves prescription of corticosteroid in combination with blood pressure medication. It can therefore be beneficial to administer to an MCD patient in need, in addition to the belatacept-like molecule, drugs that are commonly used today in the treatment of this disease.

Thus, in certain embodiments, the present invention provides the use of the belatacept-like molecule in combination with a steroid drug for use in treatment of minimal change disease. In particular examples, the steroid drug is a corticosteroid, though any steroid drug that is used for MCD treatment is contemplated for use with the described compositions.

MCD in particular, and nephrotic syndrome in general, is often associated with proteinuria, hypercholesterolemia, and edema. Therefore, the present disclosure further contemplates the use of the belatacept-like molecule in combination with one or more additional drugs currently being part of the arsenal of drugs available for the treatment of nephrotic syndrome in general, such as an angiotensin-converting-enzyme (ACE) inhibitor that can be used in place of or in combination with steroid treatment, cholesterol lowering drugs such as a statin drug and/or cyclophosphamide, or cyclosporine (the two latter particularly in elderly patients).

In certain embodiments, the belatacept-like molecule and the steroid and/or additional drug are for concomitant administration, while in other embodiments the belatacept-like molecule and said steroid and/or additional drug are for sequential administration. Consequently, in certain embodiments, the belatacept-like molecule and the steroid and/or additional drug can be in separate compositions, while in other embodiments the belatacept-like molecule and the steroid and/or additional drug can be in a single composition.

Most children suffering from MCD who are treated with steroid therapies become free of symptoms after about 8 weeks of treatment, while in adults the regimen usually includes treatment for 3 months. A large proportion of symptom-free patients (about 75%) suffer relapses of the disease and the return of symptoms. The belatacept-like molecule, alone or in combinations as defined above, can be used to treat MCD, regardless of whether it is primary and relapse MCD. Thus, in certain embodiments, the compositions are used to treat primary minimal change disease, and in other embodiments, the compositions are used to treat relapsing minimal change disease.

In one aspect, the present disclosure provides a pharmaceutical composition for the treatment of minimal change disease comprising the belataceptlike molecule and a pharmaceutically acceptable carrier, salt, excipient and the like.

In certain embodiments, the pharmaceutical composition further comprises a steroid drug.

In some instances the pharmaceutical composition may comprise an ACE inhibitor, a cholesterol lowering drug such as a statin drug and/or cyclophosphamide or cyclosporine.

The route of administration of the pharmaceutical composition is preferably systemic administration, including but not limited to, the intravenous route.

The decision whether to administer the belatacept-like molecule alone or in combination with another drug as defined above and the dosage of the agent(s) to be administered will be determined by the physician according to the agent(s), the age of the patient and phase of the disease.

In another aspect, the present invention is directed to a method for treatment of minimal change disease in an individual in need thereof, comprising administering to said individual a therapeutically effective amount of a belatacept-like molecule alone or in combination with a steroid drug and optionally in combination with an ACE inhibitor, a cholesterol lowering drug such as a statin drug and/or cyclophosphamide or cyclosporine.

The following examples are provided to illustrate certain particular features and/or embodiments. These examples should not be construed to limit the disclosure to the particular features or embodiments described.

### EXAMPLES

Example 1: Treatment of MCD in an Animal Model with Belatacept

This example shows the efficacy of belatacept in treating MCD in an animal model is tested as follows:

Ten (n=10) male Sprague-Dowley rats (weighting between 190 to 250 grams), are divided into three treatment groups:

(i) Control (no treatment): 2 rats;

(ii) 4 rats treated with a single intravenous injection of puromycin aminonucleoside in a dose of 7.5 mg/100 g; and

(iii) 4 rats treated with puromycin aminonucleoside like group B and Belatecept (Bristol-Myers Squibb, US) daily.

The rats are kept in metabolic cages that allow monitoring the rats under standardized conditions and to measure and collect urine and feces as well as food and water intake.

The study is performed over a period of 10 days, after an adaptation period of 7 days. Blood samples are drawn on days 1, 3, 6 and 10 and are examined for urea, creatinine and albumin concentrations. Urine is collected and measured daily for protein excretion, sCD80 and sCTLA-4 levels.

At the end of the study, the rats are sacrificed and the kidneys are examined by light microscopy, immunofluorescence and electron microscopy studies. Analysis of immunostained CD80 is performed on section from the kidneys. Blocking positive T cell co-stimulatory pathway with belatacept might have a therapeutic effect expressed by reduction in the urinary secretion of albumin and resolution of the fusion of foot processes in electromicroscopic evolution. Thus, a change towards normal blood urea, creatinine and albumin concentrations and towards normal urine protein, sCD80 and sCTLA-4 levels in samples collected from the treated animals, and relative to the control animals, are measured as indications of the efficacy of the treatment.

In particular, the treatment is expected to abolish the nephrotic range proteinuria and reduce the urine level of CD80. The immunofluorescence assay is expected to yield a normal result (no depositions) or non-specific mild deposition especially of IgM, which will return to normal (namely no deposition) upon treatment. Electron microscopy tests will show effacement of podocytes foot processes which will return to normal upon treatment.

Example 2: Treatment of MCD in an Human Subject This example shows the efficacy of belatacept in treating MCD in human patients.

Subjects are identified that have been diagnosed with MCD. In a particular example, subjects with primary MCD are tested. In another trial, subjects are tested who are experiencing MCD relapse following steroid treatment.

Test subjects are divided into three groups—those receiving steroid-based MCD treatment, those receiving belatacept, and those receiving a combination of belatacept and steroid treatment.

Treatments are administered intravenously on a daily basis for two weeks. Blood and urine samples are taken daily and assayed for protein levels. Urine is measured daily for protein excretion, sCD80 and sCTLA-4 levels to assess treatment efficacy. In particular, sCD80 levels are expected to be reduced in those subjects administered belatacept, and the ratio of CD80:CTLA-4 is expected to be reduced to a more normal range in belatacept recipients.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1.-12. (canceled)

13. A method for treatment of minimal change disease (MCD) in a subject in need thereof, comprising:
administering to the subject a composition comprising a molecule which binds CD80 and/or CD86, wherein the molecule comprises:
(i) a soluble extracellular domain of CTLA4;
(ii) a mutated soluble extracellular domain of CTLA4, wherein
(a) an alanine at position 29 is substituted with an amino acid selected from the group consisting of tyrosine, leucine, tryptophan, and threonine, and
(b) a leucine at position 104 is substituted with a glutamic acid; or
(iii) the soluble extracellular domain of (i) or (ii), and a moiety which alters the solubility and/or affinity to CD80 and/or CD86 of the extracellular domain of CTLA4.

14. The method of claim 13, wherein the moiety comprises a human immunoglobulin constant region.

15. The active agent of claim 13, wherein the molecule comprises the amino acid sequence of belatacept as set forth in SEQ ID NO: 1.

16. The method of claim 13 further comprising administering to the subject a steroid drug.

17. The method of claim 16, wherein the molecule and the steroid drug are administered concomitantly.

18. The method of claim 16, wherein said molecule and said steroid drug are administered sequentially.

19. The method of claim 16, wherein the steroid drug and the molecule are administered in separate compositions.

20. The method of claim 16, wherein the steroid drug is administered to the subject in the same composition as the molecule.

21. The method of claim 13, wherein the MCD is primary or relapsing MCD.

22. The method of claim 13, wherein the composition further comprises a pharmaceutically acceptable carrier, salt, or excipient.
